(19) United States
(12) Patent Application Publication
(10) Pub. No.: US 2011/0021502 A1
(43) Pub. Date: Jan. 27, 2011

(54) USE OF CHOLINESTERASE INHIBITORS FOR TREATING VASCULAR DEPRESSION

(76) Inventor: Roger Michael LANE, New York, NY (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/868,595

(22) Filed: Aug. 25, 2010

Related U.S. Application Data
(63) Continuation of application No. 12/707,691, filed on Feb. 18, 2010.
Application No. 12/707,691 is a continuation of application No. 10/597,946, filed on Sep. 1, 2006, now abandoned.
(60) Provisional application No. 60/545,871, filed on Feb. 19, 2004.

(51) Int. Cl. A61K 31/27; A61K 31/445; A61K 31/55; A61K 31/135; A61K 31/4525; A61K 31/138; A61K 31/343; A61K 31/15; A61P 9/00; A61P 25/24

(52) U.S. Cl. 514/214.02; 514/490; 514/319; 514/215; 514/479; 514/649; 514/321; 514/651; 514/657; 514/469; 514/654; 514/647; 514/656; 514/217

(57) ABSTRACT

The invention discloses the use of cholinesterase inhibitors in treating vascular depression.

This is a continuation of application Ser. No. 12/707691 filed on Feb. 18, 2010 which is a continuation of application Ser. No. 10/597,946 filed on Sep. 1, 2006, which is a National Stage of International Application No. PCT/EP2005/001715 filed on Feb. 18, 2005, which claims benefit of U.S. Provisional application No. 60/545,871 filed on Feb. 19, 2004, the entire disclosures of which are hereby incorporated by reference.

The invention relates to the use of cholinesterase inhibitors in treating vascular depression.

### FIELD OF THE INVENTION

The invention relates to the area of neurodegenerative diseases and, more particularly, to the treatment of vascular depression.

### BACKGROUND OF THE INVENTION

There is a strong relationship between cerebrovascular disease, particularly subcortical white matter lesions and depression. See Lane et al., German J Psych., Vol. 2, pp. 1-28 (1999). In addition, there is growing evidence for a sub-type of depression often—though not always—occurring in later life. Vascular damage to fronto-subcortical structures may be aetiological in late-life depression and it has also been termed “vascular depression”. See Alexopoulos et al., Am. J. Psychiatry, Vol. 154, No. 4, pp. 562-565 (1997); and Krishnan et al., Med. Hypotheses, Vol. 44, pp. 77-145 (1995). It is known that diseases affecting the subcortex are associated with high rates of depression. Vascular depression may be diagnosed from a combination of depressive ideation, greater psychomotor disturbance, apathy, executive dysfunction on neuropsychological testing, neuroimaging abnormalities in the basal ganglia and white matter on MRI. Damage to end-arteries supplying subcortical-striato-palido-thalamo-cortical pathways may disrupt neurotransmitter circuitry involved in mood regulation, thus causing or inducing depression. Potentially, this may occur either via strategically placed infarcts, particularly affecting thalamocortical projections, or is the result of an overall threshold effect. In the absence of stroke, MRI studies of patients with late-onset depression have shown white matter hyperintensities in predominantly frontal lobes and basal ganglia, of possible vascular origin. See Greenwald et al., Stroke, Vol. 29, No. 3, pp. 613-617 (1998). There is extensive overlap between the aetiology and symptoms of vascular depression and subcortical vascular dementia-prominent psychomotor retardation, cognitive impairment, especially executive dysfunction and attention deficits and apathy are seen in both disorders, in addition to psychosis and affective disturbance. See Alexopoulos et al. (1997), supra; and Moretti et al, Curr. Therapeutic Res., Vol. 63, pp. 443-458 (2002). Not surprisingly, patients with vascular depression are at risk of developing subcortical vascular dementia. See Hickie et al., Biol. Psychiatry, Vol. 42, No. 5, pp. 367-374 (1997). Patients with vascular depression, defined by the presence of subcortical white matter hyperintensities on MRI, have a poorer response to anti-depressant treatment than patients with non-vascular depression. See Simpson et al., Psychol. Med., Vol. 28, No. 5, pp. 1015-1026 (1998). In particular, cognitively impaired individuals with later life depression who respond to anti-depressant pharmacotherapy show cognitive improvements but some residual impairments, especially in memory and executive function. See Butters et al., Research and Practices in Alzheimer's Disease, Vol. 6, pp. 82-90 (2002); and Alexopoulos et al., Am. J. Psychiatry, Vol. 150, No. 11, pp. 1693-1699 (1993). The similarities between vascular depression and subcortical vascular dementia may mean that the response of depressive symptoms to rivastigmine in patients with subcortical vascular dementia [see Moretti et al. (2002), supra] suggests that rivastigmine, and possibly other cholinesterase inhibitors, may be useful as monotherapy or augmentation agents in vascular depression with evidence of subcortical vascular pathology.

Cholinesterase inhibitors including the aforementioned rivastigmine, are useful in treating a number of physiological disorders responsive to the activation of acetyl choline receptors, e.g., senile dementia, Alzheimer's Disease, Huntingdon's chorea, tardive dyskinesias, hyperkinesias, mania, acute confusion disorder, Down's syndrome, Friedrich's ataxia, multiple sclerosis and vascular dementia. However, there is nothing in the literature which teaches that cholinesterase inhibitors would be useful in treating late-onset vascular depression.

Conventional treatment of vascular depression involves the administration of anti-depressants which are utilized in treating non-vascular depression. However, although animal studies suggest that some anti-depressants promote neurological recovery after ischemic lesions, other anti-depressants inhibit recovery. Even when successful, anti-depressant therapy rarely alleviates the cognitive and executive function deficits associated with depression. More recently, drugs used in the prevention and treatment of cerebrovascular disease have been suggested to augment standard anti-depressant therapy in treating vascular depression, where early results have been mixed, at best. These include anti-cholesterinemic and anti-platelet agents, free radical scavengers, calcium channel blockers, glutamate N-methyl-D-aspartic acid receptor antagonists, gangliosides, amino-steroids and amphetamines. In view of the shortcomings of existing drugs, there is a need for a new method of treating vascular depression which alleviates the apathy, motor slowing, cognitive impairment, attention deficits and executive function deficits associated with the condition, in addition to the symptoms of depression.

### SUMMARY OF THE INVENTION

The present invention relates to the use of cholinesterase inhibitors for treating vascular depression.

In one aspect, the invention relates to the use of a cholinesterase inhibitor in the monotherapy treatment of vascular depression, preferably in the monotherapy treatment of late-onset vascular depression.

In another aspect, the invention relates to the use of a cholinesterase inhibitor in augmenting the anti-depressant therapy of vascular depression, preferably to augment the anti-depressant therapy of late-onset vascular depression.

### DETAILED DESCRIPTION OF THE INVENTION

The present invention is directed to a method of treating vascular depression utilizing a cholinesterase inhibitor.

In one embodiment, the invention is directed to a method of treating vascular depression comprising administering to a subject in need of such treatment a therapeutically effective amount of a cholinesterase inhibitor. In a preferred embodiment, the invention is directed to a method of treating late-onset vascular depression.

In another embodiment, the invention relates to the use of cholinesterase inhibitors for the manufacture of medicaments for the treatment of vascular depression, preferably late-onset vascular depression.

In another embodiment, the invention relates to pharmaceutical compositions comprising, in combination, a cholinesterase inhibitor and an anti-depressant. More particularly, said embodiment relates to a pharmaceutical composition comprising a pharmaceutically acceptable carrier or diluent and a therapeutically effective amount of: 1) a cholinesterase inhibitor; and 2) an anti-depressant.

In a further embodiment, the invention is directed to a method of treating vascular depression comprising administering to a subject in need of such treatment a therapeutically effective amount of a composition comprising, in combination, a cholinesterase inhibitor and at least one anti-depressant. In a preferred embodiment, the invention is directed to the use of said combination in treating late-onset vascular depression.

Although any cholinesterase inhibitor can be utilized in the practice of this invention, preferred cholinesterase inhibitors are those set forth in U.S. Pat. No. 4,948,807, more preferably rivastigmine tartrate; U.S. Pat. No. 4,895,841, more preferably donepezil hydrochloride; and U.S. Pat. No. 4,663,318, more preferably galanthamine hydrobromide.

For treating vascular depression, the appropriate dosage will of course vary depending upon, e.g., the compound employed, the host, the mode of administration and the nature and severity of the condition being treated. However, in general, satisfactory results are obtained when the cholinesterase inhibitor is administered in the form that it is marketed. More particularly, rivastigmine tartrate can be administered in tablet or capsule form or as a liquid oral concentrate under the tradename Exelon® in a total daily dosage of between 3 mg and 12 mg. Alternatively, rivastigmine can be administered transdermally in free base form, preferably via a transdermal patch of between 2 and 20 square centimeters (cm²). More preferably, rivastigmine can be administered at a dose of 9 mg in a patch of ˜5 cm² or at a dose of 18 mg in a patch of ˜10 cm², once every day. As regards to the other cholinesterase inhibitors, donepezil hydrochloride can be administered in tablet form under the tradename Aricept® in a total daily dosage of between 5 mg and 10 mg; and galanthamine bromide can be administered in tablet form under the tradename Reminyl® in a total daily dosage of between 12 mg and 24 mg, e.g., 12 mg twice a day.

As indicated above, the above-mentioned cholinesterase inhibitors can be utilized to augment the anti-depressant therapy in treating vascular depression. For example, they can be utilized to augment: 1) the SSRI anti-depressants, viz., paroxetine hydrochloride (Paxil®), fluoxetine hydrochloride (Prozac®), sertraline hydrochloride (Zoloft®), citalopram hydrobromide (Celexa®), etc.; 2) the SNRI anti-depressants, viz., venlafaxine hydrochloride (Effexor®), etc.; 3) the MAO inhibitor anti-depressants, viz., phenelzine sulfate (Nardil®), tranylcypromine sulfate (Parnate®), etc.; 4) the tricyclic anti-depressants, viz., amitriptyline (Elavil®), desipramine hydrochloride (Norpramin®), etc.; and 5) other anti-depressants which work somewhat differently, viz., bupropion hydrochloride (Wellbutrin®) and mirtazapine (Remeron®).

As with the cholinesterase inhibitors, the appropriate dosage of anti-depressants will, of course, vary depending upon, e.g., the compound employed, the host, the mode of administration and the nature and severity of the condition being treated. However, in general, satisfactory results are obtained when the anti-depressant is administered in the form that is marketed. More particularly, paroxetine hydrochloride (Paxil®) can be administered in tablet form in a total daily dosage of between 10 mg and 50 mg; hydrochloride (Prozac®) can be administered in tablet form in a total daily dosage of 20 mg; sertraline hydrochloride (Zoloft®) can be administered in tablet form in a total daily dosage of between 25 mg and 200 mg; citalopram hydrobromide (Celexa®) can be administered in tablet form in a total daily dosage of between 10 mg and 40 mg; escitalopram oxalate (Lexapro®) can be administered in tablet form at a total daily dosage of between 10 mg and 20 mg; venlafaxine hydrochloride (Effexor®) can be administered in tablet form at a daily dosage of between 25 mg and 100 mg; phenelzine sulfate (Nardil®) can be administered in tablet form at a total daily dosage of 15 mg; tranylcypromine sulfate (Parnate®) can be administered in tablet form at a total daily dosage of 10 mg; amitriptyline (Elavil®) can be administered in tablet form at a total daily dosage of between 10 mg and 150 mg; desipramine hydrochloride (Norpramin®) can be administered in tablet form at a total daily dosage of between 10 mg and 150 mg; bupropion hydrochloride (Wellbutrin® can be administered in tablet form at a total daily dosage of between 75 mg and 100 mg; and mirtazapine (Remeron®) can be administered in tablet form at a total daily dosage of between 15 mg and 45 mg.

Moreover, the present invention also pertains to a pharmaceutical composition comprising, in combination, a cholinesterase inhibitor and an anti-depressant, preferably a pharmaceutical composition comprising, in combination, a cholinesterase inhibitor selected from rivastigmine tartrate (Exelon®), donepezil hydrochloride (Aricept®) and galanthamine hydrobromide (Reminyl®) and an anti-depressant selected from SSRI anti-depressants, SNRI anti-depressants, MAO inhibitor anti-depressants, tricyclic anti-depressants, bupropion hydrochloride (Wellbutrin®) and mirtazapine Remeron®), more preferably a pharmaceutical composition comprising, in combination, a cholinesterase inhibitor selected from rivastigmine tartrate (Exelon®), donepezil hydrochloride (Aricept®) and galanthamine hydrobromide (Reminyl®) and an anti-depressant selected from paroxetine hydrochloride (Paxil®), fluoxetine hydrochloride (Prozac®), sertraline hydrochloride (Zoloft®), citalopram hydrobromide (Celexa®), escitalopram oxalate (Lexapro®), venlafaxine hydrochloride (Effexor®), phenelzine sulfate (Nardil®), tranylcypromine sulfate (Parnate®), amitriptyline (Elavil®), desipramine hydrochloride (Norpramin®), bupropion hydrochloride (Wellbutrin®) and mirtazapine (Remeron®), most preferably a pharmaceutical composition comprising, in combination, and an anti-depressant selected from paroxetine hydrochloride (Paxil®), fluoxetine hydrochloride (Prozac®), sertraline hydrochloride (Zoloft®), citalopram hydrobromide (Celexa®), escitalopram oxalate (Lexapro®), venlafaxine hydrochloride (Effexor®), phenelzine sulfate (Nardil®), tranylcypromine sulfate (Parnate®), amitriptyline (Elavil®), desipramine hydrochloride (Norpramin®), buptopion hydrochloride (Wellbutrin®) and mirtazapine (Remeron®).

In the above compositions, it should be understood that the cholinesterase inhibitor and the anti-depressant may be present in free form or in the form of a pharmaceutically acceptable salt together with a pharmaceutically acceptable carrier or diluent for simultaneous, separate or sequential use in treating vascular depression.

The active ingredients of the above compositions, i.e., the cholinesterase inhibitor and the anti-depressant, may also be part of a “kit” in the sense that the active ingredients can be dosed independently or by use of different fixed combinations with distinct amounts of the active ingredients, i.e., simultaneously or at different times. The parts of the kit can then, e.g., be administered simultaneously or chronologically staggered, that is at different time points and with equal or different time intervals for each component of the kit. Preferably, the time intervals are chosen such that the effect on the treated disease in the combined use of the parts is larger than the effect which would be obtained by use of only any one of the active ingredients.

Accordingly, the present invention also provides a commercial package comprising a combination as disclosed herein together with instructions for simultaneous or sequential use thereof in the treatment of vascular depression, preferably late-onset vascular depression. A preferred commercial package is one wherein one of the active ingredients is rivastigmine tartrate (Exelon®).

1. A method of treating vascular depression comprising administering to a subject in need of such treatment a therapeutically effective amount of a cholinesterase inhibitor, in free form or in pharmaceutically acceptable salt form.

2. (canceled)

3. The method according to claim 1 wherein the cholinesterase inhibitor is rivastigmine tartrate (Exelon®), donepezil hydrochloride (Aricept®) or galanthamine hydrobromide (Reminyl®).

4. The method according to claim 3 wherein the cholinesterase inhibitor is rivastigmine tartrate (Exelon®).

5. The method according to claim 4 wherein rivastigmine tartrate (Exelon®) is administered at a daily dosage of between 3 mg and 12 mg.

6. The method according to claim 1 wherein the cholinesterase inhibitor is rivastigmine which is administered transdermally in free base form.

7. The method according to claim 6 wherein rivastigmine is administered at a dose of 9 mg in a transdermal patch of ˜5 cm², once every day.

8. The method according to claim 6 wherein rivastigmine is administered at a dose of 18 mg in a transdermal patch of ˜10 cm², once every day.

9. A method of treating vascular depression comprising administering to a subject in need of such treatment a pharmaceutical composition comprising a pharmaceutically acceptable carrier or diluent and a therapeutically effective amount of: 1) a cholinesterase inhibitor; and 2) an anti-depressant, said cholinesterase inhibitor and anti-depressant being in free form or pharmaceutically acceptable salt form.

10. The method according to claim 9 wherein the cholinesterase inhibitor is selected from rivastigmine tartrate (Exelon®), donepezil hydrochloride (Aricept®) and galanthamine hydrobromide (Reminyl®).

11. The method according to claim 10 wherein the cholinesterase inhibitor is rivastigmine tartrate (Exelon®).

12. The method according to claim 11 wherein the rivastigmine tartrate (Exelon®) is present in an amount between 3 mg and 12 mg.

13. The method according to claim 9 wherein the cholinesterase inhibitor is rivastigmine in free base form and the composition is in the form of systemic transdermal pharmaceutical composition.

14. The method according to claim 13 wherein rivastigmine is present in an amount of 9 mg in a transdermal patch of ˜5 cm².

15. The method according to claim 13 wherein rivastigmine is present in an amount of 18 mg in a transdermal patch of ˜10 cm².

16. The method according to claim 9 wherein the anti-depressant is selected from selective serotonin reuptake inhibitor (SSRI) anti-depressants, serotonin-norepinephrine reuptake inhibitor (SNRI) anti-depressants, Monoamine oxidase (MAO) inhibitor anti-depressants, tricyclic anti-depressants, bupropion hydrochloride (Wellbutrin®) and mirtazapine (Remeron®).

17. The method according to claim 9 wherein the cholinesterase inhibitor is selected from rivastigmine tartrate (Exelon®), donepezil hydrochloride (Aricept®) and galanthamine hydrobromide (Reminyl®) and the anti-depressant is selected from paroxetine hydrochloride (Paxil®), fluoxetine hydrochloride (Prozac®), sertraline hydrochloride (Zoloft®), citalopram hydrobromide (Celexa®), escitalopram oxalate (Lexapro®), venlafaxine hydrochloride (Effexor®), phenelzine sulfate (Nardil®), tranylcypromine sulfate (Parnate®), amitriptyline (Elavil®), desipramine hydrochloride (Norpramin®), bupropion hydrochloride (Wellbutrin®) and mirtazapine (Remeron®).

18. The method according to claim 17 wherein the cholinesterase inhibitor is rivastigmine tartrate (Exelon®) and the anti-depressant is selected from rivastigmine tartrate (Exelon®), donepezil hydrochloride (Aricept®) and galanthamine hydrobromide (Reminyl®) and the anti-depressant is selected from paroxetine hydrochloride (Paxil®), fluoxetine hydrochloride (Prozac®), sertraline hydrochloride (Zoloft®), citalopram hydrobromide (Celexa®), escitalopram oxalate (Lexapro®), venlafaxine hydrochloride (Effexor®), phenelzine sulfate (Nardil®), tranylcypromine sulfate (Parnate®), amitriptyline (Elavil®), desipramine hydrochloride (Norpramin®), bupropion hydrochloride (Wellbutrin®) and mirtazapine (Remeron®).

19. The method according to claim 18 wherein the rivastigmine tartrate (Exelon®) is present in an amount between 3 mg and 12 mg.

20-32. (canceled)
